(19) United States
(12) Patent Application Publication
(10) Pub. No.: US 2018/0360008 A1
(43) Pub. Date: Dec. 20, 2018

(54) SYSTEM AND METHOD FOR PROPAGATING DIPTERAN LARVAE

(71) Applicants: Radu Popa, Reseda, CA (US); Kenneth H. Nealson, South Pasadena, CA (US)

(72) Inventors: Radu Popa, Reseda, CA (US); Kenneth H. Nealson, South Pasadena, CA (US)

(21) Appl. No.: 16/011,934

(22) Filed: Jun. 19, 2018

Related U.S. Application Data
(60) Provisional application No. 62/522,472, filed on Jun. 20, 2017.

(51) Int. Cl. A01K 67/033

(52) U.S. Cl. CPC A01K 67/033

(57) ABSTRACT

A method is provided for mass-rearing black soldier fly larvae (BSFL) to the mature larval stage just prior to the pre-pupal developmental stage. The BSFL biomass produced by the mass-rearing methods can be used for processing decayable biological or organic waste and converting it into protein meal for animal feeds and melanin or melanin-associated proteins.

### CROSS-REFERENCE TO RELATED APPLICATIONS

This application claims priority to and the benefit of co-pending U.S. provisional patent application Ser. No. 62/522,472, entitled “SYSTEM AND METHOD FOR PROPAGATING DIPTERAN LARVAE,” filed Jun. 20, 2017, which is incorporated herein by reference in its entirety.

### STATEMENT REGARDING FEDERALLY SPONSORED RESEARCH OR DEVELOPMENT

Not applicable

### 1. TECHNICAL FIELD

The present invention relates to systems and methods for propagating dipteran larvae. The invention also relates to systems and methods for mass-rearing black soldier fly larvae (BSFL). The invention also relates to systems and methods for mass-rearing BSFL for use in the production of protein meal for use in animal feeds. The invention also relates to systems and methods for mass-rearing BSFL for use in the production of melanin and melanin-associated proteins from fermentation leachates or from nutrient rich solutions spiked with low cost, sugar-rich sources.

### 2. BACKGROUND

Insect-based biomass can be utilized in the production of insect-derived products. The larvae of dipteran insects such as black soldier flies (BSF) can be used for the processing and disposal of food waste products. Food waste is an inexpensive source of nutrients. Fermented food waste is excellent for producing both food-waste derived leachate (“leachate”), which can be used to produce melanin, melanin-associated proteins and/or fertilizer, and fermented solid residue, which can be used to grow black soldier fly larvae (BSFL) from the first instar hatchling larval stage to the last instar prepupa. Leachate can also be added to larvae food. BSFL can be employed in methods for producing melanin, melanin-associated protein and/or inorganic fertilizers from fermentation leachates or from low-cost nutrient-rich solutions (U.S. Pat. No. 8,815,539 and WO 2014/197708 A1 by Popa et al.). The large white larvae are grown to a sufficient size to withstand, for at least two weeks (and up to 6 months), the harshness of conditions in secondary processing bioreactors for producing melanin.

According to the methods of U.S. Pat. No. 8,815,539 or WO 2014/197708 A1, polysaccharides are partly converted into natural melanins or inorganic fertilizer, which are difficult to biodegrade and hence accumulate in the bioreactors. The methods disclosed in U.S. Pat. No. 8,815,539 and WO 2014/197708 A1 can employ, as a source of nutrients, leachates produced from food waste and sugars-rich liquid waste of the food industry. These leachates can be used raw or they can be augmented with low-cost sugar-rich solutions such as molasses, hydrolyzed cellulose or starch. These methods are inexpensive and do not require the use of expensive chemically-defined culture media.

Melanin

The methods disclosed in U.S. Pat. No. 8,815,539 and WO 2014/197708 A1 use BSFL to produce melanin. Melanin is one of a very few known examples of natural organic semiconductors and was demonstrated to be such in the early 1970s. Melanin is thus a desirable natural, environmentally friendly material with many known applications for the electronics industry. Melanin can be used to produce a wide variety of biologically friendly electronic devices and batteries used in applications such as medical sensors and tissue stimulation treatments.

Protein Meal

The world's needs for animal-derived protein products (such as fish meal, soymeal and peanut meal) in animal feed and aquaculture applications are growing rapidly. The needs for fish meal are outstripping the ocean's ability to regenerate the forage fish that are harvested to manufacture fish meal. Precipitous and unsustainable decline in ocean fish stocks put strain on aquaculture and poultry culture. Vegetal sources of proteins, such as soy meal and peanut meal, are in limited supply because they are costly to produce and because they are usable as human food. Growing vegetal biomass (such as soy and peanuts) to feed livestock is not sustainable when these materials can be used to feed humans directly.

Insect-based biomass can be utilized in animal feeds as a substitute for animal-derived proteins (such as the proteins in fish meal) or plant-derived proteins (such as the proteins in soy meal or peanut meal). One possible approach for insect rearing is to utilize pre- and post-consumer food waste and other biological wastes as feedstock. Cultured invertebrates, such as earthworms, meal worms, shrimps, prawns or crayfish, crickets and fly larvae, that can be used to feed humans or animals, or to make fertilizers, can be fed with food waste and other biological wastes or derivatives of food waste and other biological wastes.

According to the United Stated Environmental Protection Agency, using food waste and other biological wastes to feed invertebrates is classified as an industrial use and is preferable to composting or landfilling (http://www.epa.gov/foodrecovery/, last visited Feb. 18, 2017). However, the collection, transportation and storage of unprocessed food waste before it is fed to animals (including invertebrates) can lead to many problems. These problems include fast rates of putrefaction of the food waste, the release of decaying liquids and odors, the propagation or multiplication of food-borne pathogens, the production of food-borne toxins hazardous to humans and livestock, the production of toxic molds and chemicals that are toxic for microorganisms and invertebrates and the attraction of vermin.

If there is an asymmetry between supply and demand, excess food waste becomes a health hazard and must be landfilled, composted or burned, all of which are inferior as uses to the industrial generation of animal feed products. Hence, decaying biological waste (which, as used herein, is organic waste, food waste, or other biologically-derived waste) must be collected and processed efficiently as it is generated.

Feeding urban food waste (whether raw or processed) directly to livestock increases the risk of spreading food-borne pathogens and is not efficient in industrial animal farms. Food waste, especially post-consumer urban and domestic food waste, typically contains materials that are un-digestible or hazardous to animals (including plastic, paper, cutlery, spices, wax, etc.). Using food waste as fertilizer (whether raw or processed) only works in limited cases. Many types of food waste and food waste derivatives are rich in food preservatives (such as sodium and chloride) that are undesirable for plant growth. Raw or insufficiently mineralized food waste increases the risk of spreading microbial pathogens and of molding in soil with release of poisonous chemicals such as aflatoxins.

Citation or identification of any reference in Section 2, or in any other section of this application, shall not be considered an admission that such reference is available as prior art to the present invention.

### 3. SUMMARY OF THE INVENTION

A method is provided for mass-rearing dipteran larvae to the mature larval stage just prior to the pre-pupal developmental stage. In one embodiment, the dipteran larvae are black soldier fly larvae (BSFL). The dipteran biomass produced by the mass-rearing methods disclosed herein can be used for processing decayable biological waste or organic waste, and for converting it into live feed (i.e., live larvae), feed comprising dried larvae (“dried larvae feed”) or defatted protein meal. Such feeds can be used as animal feeds or in the production of melanin or melanin-associated proteins.

A method for mass-rearing dipteran flies is provided comprising:

providing:
a larval incubation container,
inert substrate material,
a larval feed,
dipteran fly larvae, and
water;
grinding and/or sieving the inert substrate material to obtain substrate material particles having largest diameters of ≤3.2 mm;
grinding and/or sieving the larval feed to obtain larval feed particles having largest diameters of ≤3.2 mm;
drying the inert substrate material;
adding dry inert substrate material to the larval incubation container;
adding the larval feed to the larval incubation container;
mixing the larval feed and the dry inert substrate material in the larval incubation container;
adding the dipteran fly larvae to the larval incubation container;
adding water to the dipteran fly larvae, the larval feed and dry inert substrate material in the larval incubation container to obtain a ratio, wherein the ratio ranges by weight from 1.5 parts water:1 part dry inert substrate material plus the larval feed to 2.5 parts water: 1 part dry inert substrate material plus the larval feed;
incubating the dipteran larvae in the larval incubation container; and
harvesting the dipteran flies produced in the larval incubation container.

In an embodiment of the method, the substrate material particles and/or the larval feed particles have largest diameters of ≤2 mm. In another embodiment of the method, the largest diameters of the substrate material particles and/or the larval feed particles are between 0.1 mm and 2 mm. In another embodiment of the method, the largest diameters of the substrate material particles and/or the larval feed particles are between 2 mm and 3.2 mm. In another embodiment of the method, the largest diameters of the substrate material particles and/or the larval feed particles are between 3.2 mm and 3.4 mm

In an embodiment of the method, the dipteran flies are black soldier flies (BSF).

In an embodiment of the method, the harvested dipteran flies are larvae.

In an embodiment of the method, the harvested dipteran flies are pre-pupae.

In an embodiment of the method, the harvested dipteran flies are pupae.

In an embodiment of the method, the harvested dipteran flies are adults.

In an embodiment of the method, the inert substrate material is a ligno-cellulosic material.

In an embodiment of the method, the dipteran larvae added to the larval incubation container are black soldier fly larvae (BSFL) that are 2-4 days old and weigh about 5-30 mg wet weight per larva.

In an embodiment of the method, before the harvesting of the dipeteran fly larvae, the dipteran fly larvae are starved for 2-4 days.

In an embodiment of the method, after the harvesting of the dipeteran fly larvae, the harvested fly larvae are placed in water and starved for approximately 24-48 hours.

In an embodiment of the method, the larval feed is a low-fat larval feed having a fat content of 5-15%.

A system for mass-rearing dipteran flies is also provided. In an embodiment, the system comprises:

a larval incubation container,
dry inert substrate material, wherein the dry inert substrate material comprises substrate material particles, wherein the largest diameters of the substrate material particles are ≤3.2 mm a larval feed,
dipteran fly larvae, and
means for separating the dipteran fly larvae from the substrate material.

In an embodiment of the system, the largest diameters of the substrate material particles and/or the larval feed particles are between 0.1 mm and 2 mm. In another embodiment of the system, the largest diameters of the substrate material particles and/or the larval feed particles are between 2 mm and 3.2 mm. In another embodiment of the system, the largest diameters of the substrate material particles and/or the larval feed particles are between 3.2 mm and 3.4 mm

### 4. BRIEF DESCRIPTION OF THE DRAWINGS

The present invention is described herein with reference to the accompanying drawings, in which similar reference characters denote similar elements throughout the several views. It is to be understood that in some instances, various aspects of the invention may be shown exaggerated, enlarged, exploded, or incomplete to facilitate an understanding of the invention.

FIG. 1. Flow diagram showing one embodiment of the method for propagating dipteran larvae disclosed herein.

FIG. 2. System for propagating dipteran larvae, for example, black soldier fly larvae (BSFL). The system comprises a pupae harvesting collection box for harvesting pupae (6). Systems for propagating dipteran larvae can be scaled up or down to suit a wide range of dimensions for larval incubation containers and production schemes. Larval incubation container (1). Optional mosquito net (2) to limit access of nuisance arthropods (e.g., insects) such as gnats and houseflies to the container. Aeration fan (3). Feed mixture (4). Optional larvae harvesting funnel (5). Pupae harvesting collection box (6).

FIG. 3. Dipteran fly nursery for producing and harvesting dipteran eggs. Nurseries can be scaled up or down to suit a wide range of production schemes and can have, in embodiments, controlled environmental conditions. The nursery is constructed with screen to hold the flies and BSF larvae (BSFL) inside (1), and contains adult BSF (2), larvae feeding containers (3), egg laying substrates (4), water source (5), and collectors for larvae at the pre-pupal stage (“black larvae collectors”) (6). Not shown in this diagram are additional elements that can, in embodiments, be employed such as temperatures controllers, moisture controllers, air purifiers or traps for nuisance arthropods (e.g., insects) such as gnats, ants and houseflies.

FIG. 4. Diagram of larval incubation container for mass-rearing dipteran larvae such as black soldier fly larvae (BSFL). The larval incubation container may vary in dimensions. Panel (A) is an end view cross section of the larval incubation container. Panel (B) is a side view cross section of the larval incubation container with mosquito net removed and showing a mixer/spreader/rake device seen from the side. Panel (C) is a top view of the larval incubation container showing a mixer/spreader/rake device seen from top. Aeration fan (1). Larvae growth (incubation) container (2). Side rail (3). Mosquito net (4). Larvae bedding (5). Larvae (6). Mixer, spreader, surface rake device (7). Wheel (8). Blade angle adjuster (9). Plow/mixer blades (10), which can vary in number and shape. Rake (11). Chassis axis (12).

FIG. 5. An overview of the methods disclosed in U.S. Pat. No. 8,815,539 and WO 2014/197708 A1, which are used to convert media rich in sugars and polysaccharides (comprised in primary leachate) in solutions with high concentration of melanin, including but not limited to pyomelanin (comprised in secondary leachate).

FIG. 6. Diagram showing exemplary uses for the systems and methods for propagating dipteran larvae. The method can be used to mass-rear large numbers of dipteran larvae that can be used to convert raw materials (e.g., food waste, residues of food industry, gardening and agriculture vegetal waste) into live larvae biomass, larvae feed, dried larvae, protein meal and melanin.

### 5. DETAILED DESCRIPTION OF THE INVENTION

Methods and systems are provided for mass-rearing dipteran larvae black soldier fly larvae (BSFL), to the mature larval stage just prior to the pre-pupal stage.

The biomass produced by this mass-rearing can be used to produce protein meal used for animal feeds or to produce melanin or melanin-associated proteins.

In one embodiment, the method for mass-rearing dipteran flies comprises:

providing:
a larval incubation container,
inert substrate material,
a larval feed,
dipteran fly larvae, and
water;
   grinding and/or sieving the inert substrate material to obtain particles having largest diameters of ≤3.2 mm;
   grinding and/or sieving the larval feed to obtain particles having largest diameters of ≤3.2 mm; drying the inert substrate material;
   adding dry inert substrate material to the larval incubation container;
   adding the larval feed to the larval incubation container;
   mixing the larval feed and the dry inert substrate material in the larval incubation container;
   adding the dipteran fly larvae to the larval incubation container;
   adding water to the dipteran fly larvae, the larval feed and dry inert substrate material in the larval incubation container to obtain a ratio, wherein the ratio ranges by weight from 1.5 parts water:1 part dry inert substrate material plus the larval feed to 2.5 parts water:1 part dry inert substrate material plus the larval feed;
   incubating the dipteran larvae in the larval incubation container; and
   harvesting the dipteran flies produced in the larval incubation container.

This ratio of water:dry inert substrate can be calculated using methods known in the art. One needs to know the approximate moisture content of the dry inert substrate material plus larval feed. This is simple to determine using art-known methods. In one embodiment, the approximate moisture content of the dry inert substrate material is 5-6% moisture and the approximate moisture content of the larval feed is also 5-6% moisture.

The method has many advantages over current methods employed for mass-rearing dipteran insects. The method requires little labor and is amenable to automation. The method minimizes odor. The method greatly decreases or prevents larval mortality caused by mold contamination. The method avoids contamination of the BSFL culture with houseflies, gnats or other nuisance arthropods (e.g., insects). The method also maximizes the food-to-larvae biomass conversion efficiency. The method has a high larvae yield. The method has low water consumption.

For clarity of disclosure, and not by way of limitation, the detailed description of the invention is divided into the subsections set forth below.

5.1. Black Soldier Flies (BSF)

The methods and systems disclosed herein can be used for mass-rearing dipteran flies. Any dipteran fly known in the art can be used in the disclosed methods and system. In an embodiment, a fly species in the family Stratiomydae is used.

In one embodiment, black soldier flies (BSF), Hermetia illucens, are used. BSF are insects in the order Diptera. BSF are ubiquitous throughout much of the world extending between roughly the equator and 45th degree latitude (Newton et al., J. Anim Sci., 44:395-400, 1977; Bondari and Sheppard, Aquaculture and Fisheries Management, 18:209-220, 1987; Sheppard et al., Bioresource Technology, 50:275-279, 1994; Tomberlin et al., Ann. Entomol. Soc. Am., 95:379-386, 2002; St-Hilaire et al., J. World Aquaculture Society, 38:59-67, 2007; St-Hilaire et al., J. World Aquaculture Society, 38:309-313, 2007). They are holometabolous insects that undergo a life cycle of complete metamorphosis. The life cycle progresses from the egg to hatching of the larva, typically 5-6 larval instars or stages, pre-pupa, pupation into the pupal stage, then ecdysis (emergence) into the adult fly. During the pre-pupa stage, between the larva and pupal stages, the larva enters the wandering stage, moves away from or out of the nutrient source to find a pupation site on a dry surface or inside an aerated crevice. The skilled artisan is familiar with larvae generally, and with methods of breeding and propagating larvae. For example, methods of breeding and propagating dipterans including Hermetia illucens larvae can be found, e.g., in Fatchurochim et al., J. Entomol. Sci., 24:224-23, 1989.

Earlier methods of breeding and propagating black soldier fly larvae (BSFL), including methods of breeding BSFL in captivity, as well as methods for using BSFL to process solid wastes and organic leachates, are familiar to the skilled artisan (see for example, Tomberlin et al., Environ Entomol., 38 (3):930-4, 2009; Sheppard et al., J. Med. Entomol., 39 (4):695-8, 2002; Tomberlin, J. Econ. Entomol., 95:598-602, 2002; U.S. Pat. No. 6,780,637; US 2012/0187041; Popa et al., J. Econ. Entomol., 105(2): 374-378, 2012). These earlier methods, however, are inefficient and do not yield high enough BSFL biomass to be commercially viable methods. These earlier methods are based on the property of the larvae to self-harvest when they reach maturity. In these methods, the late harvest leads to a food conversion ratio that is large and food usage efficiency that is low.

The methods disclosed herein, by contrast, comprise harvesting the larvae early (during or at the end of the exponential growth phase). This results in very good food usage efficiency (with food conversion ratio between approximately 0.8 and 1.1)

An initial supply of BSFL for use in the propagation and mass-rearing methods disclosed herein can be purchased commercially, for example BIOGRUBS™ BSFL (Prota Culture, LLC, Dallas, Tex.) and PHOENIX WORMS™ BSFL (Insect Science Resource, LLC, Tifton, Ga.). Alternatively, to obtain an initial supply of BSFL, BSFL or eggs laid by adults can be harvested in the wild by gathering eggs and larvae present in animal manure, particularly chicken and pig manure, on farms and at commercial animal facilities open to the elements, especially in warmer climates where the insects are known to lay eggs throughout the year in the wild.

BSF eggs take approximately 4-6 days to hatch at 30° C. and are typically deposited in crevices or on surfaces above or adjacent to the food source. BSFL approaching the pupae stage reach a size more than 2 cm in length and 0.4 cm in diameter relative to immature larvae, which start out on hatching from eggs at less than 0.2 cm in length and less than 0.1 cm in diameter. Although larvae can be stored live at room temperature for several weeks, their longest shelf life (4-6 weeks) is achieved at 50-60° F. (10-16° C.) and when food is present but limiting.

BSFL feed on a variety of vegetal and manure wastes of varying extreme pH ranges and O₂ tensions, self-harvest on entering the pupae stage from the organic matter they are feeding on, and are ubiquitous throughout much of the world, extending between roughly the equator and 45th degree latitude (Newton et al., J. Anim Sci., 44:395-400, 1977; Bondari and Sheppard, Aquaculture and Fisheries Management, 18:209-220, 1987; Sheppard et al., Bioresource Technology, 50:275-279, 1994; Tomberlin et al., Ann. Entomol. Soc. Am., 95:379-386, 2002; St-Hilaire et al., J. World Aquaculture Society, 38:59-67, 2007; St-Hilaire et al., J. World Aquaculture Society, 38:309-313, 2007). BSFL consume organic matter, including kitchen waste, spoiled feed, and manure, and assimilate organic compounds in the organic matter into larva biomass. BSFL, like most dipteran insects, wander out of, or “self-harvest” from, the organic matter they are feeding on upon entering the pupal stage. Adult BSF do not eat; they survive for approximately one week on the fat stored from the larva stage.

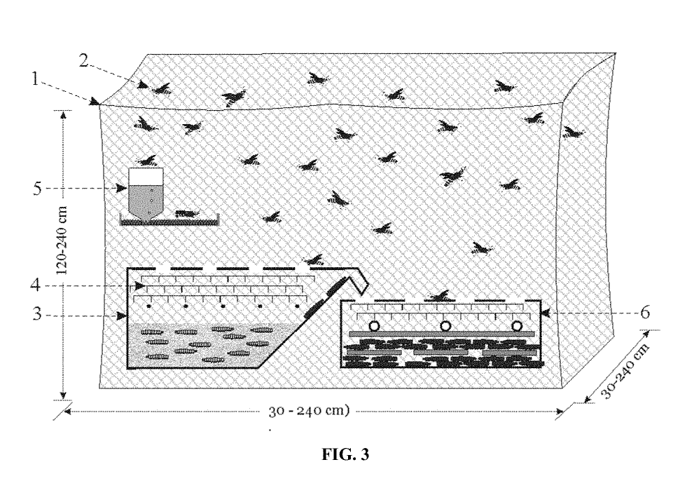

Prior to the introduction of BSFL into the mass-rearing methods, as described hereinbelow, BSFL can be hatched from egg clutches laid by mating adult flies in an insect nursery or incubator (FIG. 3) set up for rearing flies on decomposing vegetal and food scrap residues (banana peelings, leftover fragments of lettuce, stale bread, rotting tomatoes, apples, and other discarded produce, yard debris including mowed grass clippings, etc.) under conditions known in the art (Tomberlin et al., Environ Entomol., 38 (3):930-4, 2009). The eggs can be hatched in wheat bran moistened with tap water. At approximately 4 days to 1 week of age, larvae can be freed from the growth substrates along with the wheat bran by washing them in stainless steel mesh colander or fine fabric (mesh size approximately 0.05-0.1 mm) with several liters of tap water, for example, for 30 minutes to 1 hour.

FIG. 3 is a cross-sectional diagram of an embodiment of a black soldier fly (BSF) nursery used to produce and harvest eggs of BSF for use in the mass-rearing method disclosed herein. Nurseries can be scaled up or down to suit a wide range of production schemes and can have controlled environmental conditions. The nursery is constructed with screen to hold the flies and BSF larvae (BSFL) inside (1), and contains adult BSF (2), larvae feeding containers (3), egg laying substrates (4), water source (5), black larvae collectors (6). Not shown in FIG. 3 are additional elements that can be employed in various embodiments, such as temperatures controllers, moisture controllers, air purifiers and traps for gnats and houseflies.

The color of BSFL larvae is cream or ivory, until a few days before the larvae turn into pre-pupae, when the larvae start to accumulate black pigments and turn gray, then dark grey, they eventually black. This stage just before the pre-pupal stage is referred to herein as black larvae. The black larvae will still eat for 1-2 more days but will eventually stop eating and try to migrate out in a dry place to become pupae (also black in color).

5.2. Method for Mass-Rearing Black Soldier Fly Larvae (BSFL)

A method for mass-rearing black soldier fly larvae (BSFL) is provided that avoids allowing the larvae to mature to the last instar and produces larvae with low fat content and high protein:fat ratios. Allowing larvae to mature to the last instar can be very feed inefficient. The method provided herein allows for harvesting the larvae early, at a stage when their protein content is high (≥54%), their fat content is low (≤18%), and when the larvae do not contain much carbonate in their cuticle (<50%). In normal larvae, about 15-25% of the dry weight is the hard cuticle the larvae gain in the last instar, half of which is mineral carbonate. The method provided herein also allows for harvesting the larvae at a stage when the food conversion ratio (FCR) is best (between 0.9 and 1.1, depending on the quality of the feed source).

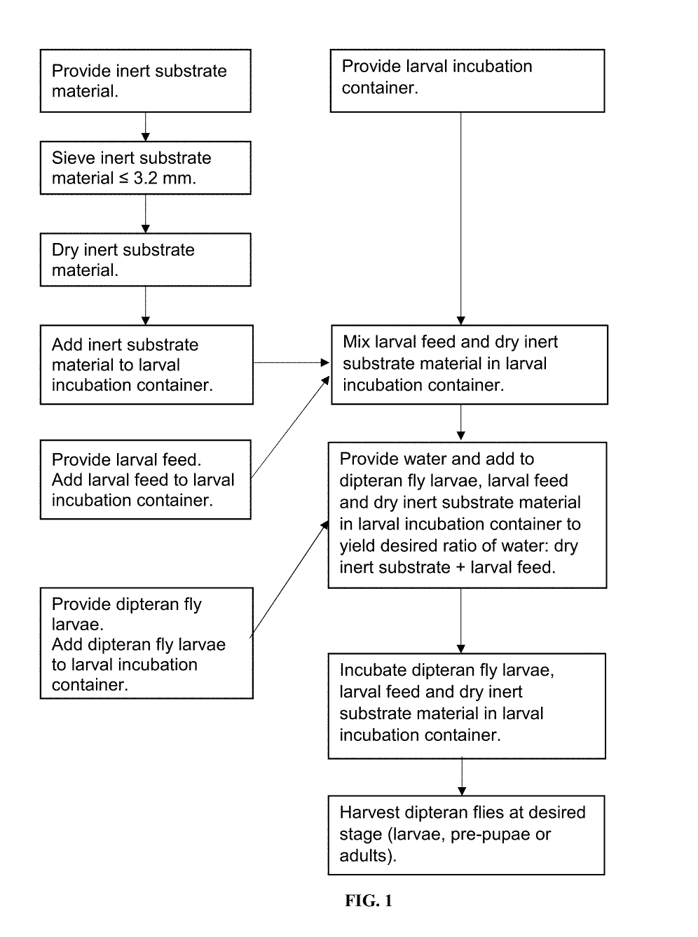

FIG. 1 shows a flow diagram of one embodiment of the method for propagating dipteran larvae disclosed herein. In one embodiment, the method for mass-rearing black soldier fly larvae (BSFL) comprises the following.

Providing a Larval Incubation Container for Mass-Rearing

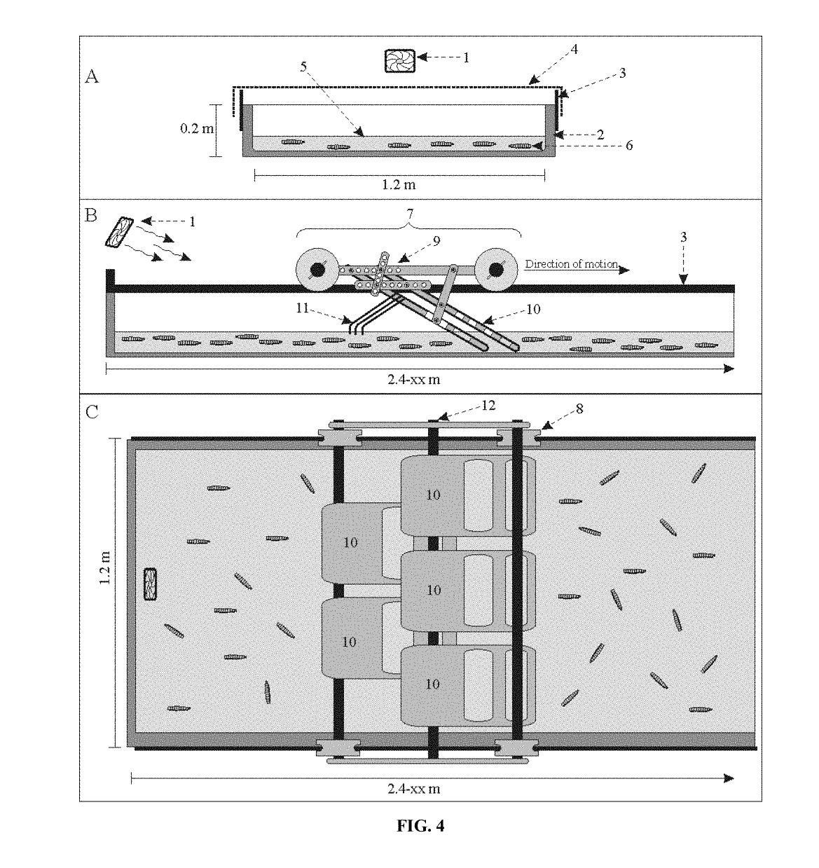

A larval incubation container is provided in which the BSFL will be mass-reared. FIG. 4 shows two cross-sectional diagrams and a top view of an embodiment of a larval incubation container for mass-rearing black soldier fly larvae (BSFL). The larval incubation container may vary in dimensions. FIG. 4, panel (A) shows an end view cross section of the larval incubation container. FIG. 4, panel (B) shows a side view cross section of the larval incubation container with mosquito net removed and showing a mixer/spreader/rake device seen from the side. FIG. 4, panel (C) shows a top view of the larval incubation container showing a mixer/spreader/rake device seen from top. Aeration fan (1). Larvae growth (incubation) container (2). Side rail (3). Mosquito net (4). Larvae bedding (5). Larvae (6). Mixer, spreader, surface rake device (7). Wheel (8). Blade angle adjuster (9). Plow/mixer blades (10), which can vary in number and shape. Suitable numbers and shapes of blades can be determined by the skilled artisan. Rake (11). Chassis axis (12).

In one embodiment, recommended for small scale operations a plastic container obtained commercially having dimensions of 51 cm long*35 cm wide*17 cm high (surface area 0.18 m²) is used. In one embodiment, the larval incubation container is 1.2 m wide, 2.4 m long and 0.2 m high and it is lined with food safe materials such as EPDM or fiberglass. In one embodiment, the larval incubation container is as long as 25 m. In one embodiment, the side walls of the larval incubation container are smooth, the top part of the bedding material is kept dry, the sides are vertical and 15-25 cm tall and aeration is provided from small fans; this combination of factors limit larvae from migrating out of the container during the wandering larval phase. In an embodiment, the container can be covered with a lid with a fine net or screen (e.g., mosquito netting or any other suitable netting with pore sizes 0.1-0.5 mm). The net helps block contamination of the culture with houseflies and gnats, which is common in warm seasons and when food is in excess relative to what the BSF larvae can eat. When using a small-scale larval incubation container, for example <1 m² totes, the empty larval incubation container can be weighed before adding inert substrate material to determine the starting weight before mass-rearing of the BSFL begins. This starting weight can be used later to determine the amount of BSFL biomass produced in the larval incubation container.

Providing an Inert Substrate Material

An inert substrate material (also referred to herein as “larvae bedding”) is provided for mass-rearing dipteran fly larvae in the larval incubation container (FIG. 4). Larvae bedding materials include, but are not limited to, ligno-cellulosic materials such as sawdust, wood flour, ground straw, ground hay, and the like.

In one embodiment, the particles of the inert larvae bedding material have largest cross-sectional diameters of between 0.1 mm-3.2 mm in diameter. In one embodiment the bedding and feed particles are pre-sorted with a ≤2 mm sieve. To produce such particle dimensions, the inert substrate material can be ground if it is initially coarser than having particle dimensions not larger than 3.2 mm. Any grinder or mill known in the art suitable for grinding ligno-cellulosic or similar materials can be used, such as a grain mill set on a coarse grind setting. In other embodiments, inert substrate material can be obtained (or ground) that has particle diameters of 0.01 mm to 3.2 mm Particles that are too small result in lots of dust and results in growth mixtures that are sticky and make larvae difficult and to separate from the bedding. Particles that are larger than 3.2 mm (˜⅛ inch) can lead to difficulties in producing clean larvae by sieving and produce protein meal with a high proportion of lignocellulose fibers. Separation of the larvae from the bedding can be accomplished using any suitable separating means known in the art, such as a sieve, and reduces odor from amines, sulfides, thiols and other odoriferous substances.

In an embodiment, separation of the larvae from the bedding and cleanest larvae are obtained when particles of bedding and food are separated with a 2 mm pore-size sieve, while the larvae are separated from the growth mixture (bedding and food) with a 3.2 mm (i.e. ⅛ inch) pore-size sieve.

Sieving the Inert Substrate (“Larvae Bedding”) Material

Once the desired particle diameter is obtained, the inert substrate material is sieved through a sieve. In an embodiment, the inert substrate material is sieved through a sieve with a pore size of approximately 3.2 mm (or about ⅛ inch) to obtain particle diameters of 3.2 mm or less. In one embodiment, the inert substrate material is sieved at least twice, which helps to eliminate long fibers (e.g., cellulosic fibers) from the mixture. In an embodiment, the inert substrate material particle diameters are 2 mm or less and the particles are separated using a sieve with ≤2 mm pore size.

In one embodiment, small particles of the inert substrate material (“dust,” i.e., particles of less than 0.2 mm in diameter) are eliminated from the inert substrate material. This can be done by sieving with a finer sieve, with or without an air stream. Elimination of these very fine particles helps produce growth mixtures that are less sticky, thus lowering the cost of larvae separation and improving product quality. If recycled, the cellulosic substrate should be sterilized and sieved before it is used, in order to kill cells, eggs and larvae of other flies, gnats and other contaminating organisms and microorganisms.

Drying the Inert Substrate Material

The inert substrate material (also referred to herein as larvae bedding or “cellulosic substrate”) is dried or dehydrated to eliminate excess water. In one embodiment, drying is performed by exposing the inert substrate material to a dry air flow at 55-95° C. In an embodiment, the substrate is reused without drying or sterilization.

Adding Dry Inert Substrate Material to the Larval Incubation Container

Dry inert substrate material is added to the larval incubation container. In an embodiment, 1 kg of dry cellulosic inert substrate material is added to the empty larval incubation container. In this embodiment, the density of the cellulosic inert substrate material is 0.2 g/ml when the moisture level is about 5-10%. In other embodiments, the density of dry inert substrate material is approximately 0.3 g/ml when the moisture level is about 10-40%. In another embodiment, 8-12 kg of dry inert substrate material is added for every square meter of the surface area of the larval incubation container.

Adding Larval Feed to the Larval Incubation Container and Mixing the Larval Feed with the Dry Inert Substrate Material.

In an embodiment, a known amount of a larval feed (dry or wet) is added to the larval incubation container. In an embodiment, the larval feed contains the equivalent of about 50 g of dry weight feed. The amount of water in the larval feed should be known to adjust the moisture level of the final mixture. The larval feed can be any feed suitable for dipteran fly rearing known in the art, and can include, but is not limited to, fermented food waste, liquids from food fermentation, ground food waste, vegetal material such as leaves, bacterial biomass from incubators or fermenters, brewers' grain, beer mash, fruit pulp, and the like). The larval feed is mixed with the inert substrate material.

In an embodiment, the larval feed is pre-sieved through a 2 mm pore-size sieve. In another embodiment, the larval feed is ground and/or sieved to obtain larval feed particles having largest diameters of ≤3.2 mm. In another embodiment, the largest diameters of the larval feed particles are between 0.1 mm and 2 mm. In another embodiment, the largest diameters of the larval feed particles are between 2 mm and 3.2 mm. In another embodiment, the largest diameters of the larval feed particles are between 3.2 mm and 3.4 mm

Adding BSFL to the Larval Incubation Container

BSFL are then added to the larval incubation container. Small BSFL can be produced in an insect nursery or incubator as described above. The larvae added should be approximately 2-4 days old and weigh about 5-30 mg wet weight per larva. If the larvae are too small they are vulnerable and many of them will die after transfer. If larger larvae are kept in the hatching containers the large larvae will cannibalize eggs and newborn larvae. Larvae produced in an insect nursery or incubator can be transferred into the larval incubation container by gently washing them into the container with a light water shower, mist, stream or by dipping the egg holders with larvae in a water reservoir. In an embodiment, the larvae are washed into a temporary water reservoir. In an embodiment, the water from the reservoir and the water from the shower are at a temperature of 25-30° C. to protect the larvae from thermal shock. Washing usually takes no longer than 30-60 minutes.

Generally, mass-rearing is less efficient if newly hatched larvae (<1 day old) are added to the larval incubation container. Newly hatched larvae are very delicate and many of them will die under the incubation conditions in the larval incubation container, which lowers the efficiency of the method.

Adding Water to the Larval Incubation Container

The larval incubation container contains the inert substrate material, larval feed, larvae and water. In one embodiment of the method, the larval incubation container is weighed. Additional water is added until a ratio between water and the dry materials (i.e., the dry inert substrate material plus feed) of approximately 1.5:1 to 2.5:1 is obtained. In one embodiment, water is gently added to the larval incubation container using a shower head or mister. Adding less water than the amount to obtain the ratio of 2.0 (water):1 (dry materials) will not leave sufficient water in the pores of the inert growth substrate for the larvae. Adding more water than the amount to obtain the ratio of 2.5 (water):1 (dry materials) will result in a muddy mixture with clogged pores in the inert growth substrate and poor aeration, and many larvae will die of suffocation. The mixture between the inert growth substrate and water is mixed thoroughly, yet carefully to avoid crushing the young BSFL, which are delicate and sensitive to injury.

The density of the mixture of water with the moistened inert substrate material and larval feed can also be determined. In an embodiment, the density is 0.35-0.55 g/ml. In another embodiment, the density is 0.65 g/ml or below; any higher, and the mixture will clog the pores of the inert growth substrate, water will pool at the bottom of the container and make the moistened inert substrate anaerobic. In one embodiment, the mixture of water with the moistened inert substrate material and larval feed feels a bit heavy, moist and fluffy to the touch, like the consistency of steamed rice or rice pilaf (i.e., it is well aerated), rather than muddy (or has the consistency of wet heavy mud). The mixture should leave wet fingers but drip little water when squeezed between the fingers and no water should be pooling or collecting on the bottom of the larval incubation container. In one embodiment, the mixture is approximately 7 cm deep in the larval incubation container. In another embodiment, the mixture is approximately 4 to 15 cm deep.

To control mold, the surface of the mixture in the larval incubation container can be misted with about 10-100 ml of a saturated solution of methyl paraben per square meter of the larval incubation container. Other mold preventative solutions known in the art can also be used, such as prior drying and sterilization of the larval incubation container. The larval incubation container is then closed with the lid with the net insert.

Incubating the BSFL in the Larval Incubation Container

The BSFL are incubated at 25-35° C. for about 24 hours. In one embodiment, the temperature maintained close to 30-32° C. At incubation temperatures lower than 25° C., larval growth will slow, and the lower temperature will promote the growth of molds. Incubation temperatures higher than 35° C. will result in fast decay and anoxia, especially if too much feed has been added. Exposure of the larval incubation container and the BSFL within it to direct light should be avoided, if possible, during incubation. A fan may be placed near the lid to aerate the surface of the mixture in the larval incubation container. A strong air flow should also be avoided, to prevent dehydrating the mixture. The objective of aeration is to provide oxygen, to keep molds under control and to create a layer of dry mixture on top which will limit larvae from migrating up and escaping from the larval incubation container. In an embodiment, the moisture content in the larvae incubation room is approximately 40-60% to help create and maintain a dry layer on top of the larval incubation container.

After approximately 1 day of incubation, the weight of the larval incubation container is determined to evaluate water loss. This evaluation of water loss is performed daily or once every two days. Water and feed can be added. In one embodiment, 50 g dry weight equivalent of feed is added daily, along with water to maintain the ratio of 2.5 (water):1 (dry materials). The loss of water by evaporation will be approximately 0.5-0.6 kg/day per 0.18 m² container at 35° C. in a room with air at a relative humidity of 40-60%.

After another day of incubation, water loss, the state of the larvae and feed availability is evaluated again. The BSFL may double in size per day (depending on the growth conditions and feed availability and quality). The amount of feed added every day is about double the amount added the day before. If BSFL are too numerous, growth is slow and more feed has to be added. For good growth, the density of larvae in growth containers is approximately 30,000-40,000 larvae per square meter. The largest density of larvae tested where good production was nonetheless obtained was 135,000 larvae per square meter. If the amount of feed is too great relative to the capacity of the larvae to consume that amount in one day, the mixture will smell of decay. If too much feed was added the days before, the incubation mixture can be mixed thoroughly 3-4 times a day by hand or by using a mixer/spreader/rake device (FIG. 4), aerated more, or no additional feed can be added for one or more days. Throughout the incubation, the water to dry mixture ratio is maintained at about 2.5:1.

After about 3-4 days when the BSFL have grown larger (about 40-100 mg), more water is added to reach a water: mixture (dry inert substrate plus larval feed) ratio of about 3:1 (rather than 2.5:1) and a density of about 0.45-0.55 g/ml. The material should not be allowed to evaporate to below 0.4 g/ml during the incubation period. The mixture can be mixed thoroughly at least twice a day to dislodge clumps, to disperse feed and moisture and to evaluate the state of the BSFL.

BSFL are creamy white or ivory in coloration until the last instar of their life cycle. After about 7-8 days, when the BSFL are approximately 100-150 mg in weight, no (or a very few) black pre-pupal stage BSFL are seen.

During the last instar, a few days before they turn into pre-pupae, the BSFL start accumulating black pigments and turn gray, then dark gray, then eventually black. This is their natural pigmentation and appearance. Pre-pupal stage BSFL are thus identified by their black appearance. BSFL that are black will still eat for 1-2 more days but will eventually stop eating and try to migrate out of the substrate in which they are living and feeding to a dry place to pupate. The pupae are also black in color.

Incubating longer will produce an excessive abundance of black BSFL, which are unusable to make melanin because they are at a developmental stage where they stop feeding and turn into pupae. In an embodiment of the method, feeding the BSFL stops about 2-3 days before harvesting the BSFL to allow the BSFL to consume or use up most of the feed. This period of larvae starvation also helps lower the smell of decay in larval incubation container. The combination between harvesting the larvae biomass in the white larvae stage, starving the larvae for about two days prior to larvae biomass harvesting and washing the harvested larvae for approximately 24-48 hours (1-2 days) in water results in larvae with lower fat content (as low as 18% fat). During the last two days of starvation, water may still be added as needed to compensate for water loss by evaporation, but generally the objective is to lower the water content until the density of the growth substrate is approximately 0.3 g/ml and less than 0.35 g/ml. In the last two days of starvation period it is also recommended to mix the larvae bedding containing the larvae thoroughly a few times a day. This will produce a mixture free of clumps that is easier to separate.

Thus, the methods disclosed herein can be modified to produce larvae with abnormal or unnatural proportions of protein and fat. To produce such larvae, the fat content of the larval feed can be modified, and/or the larvae can be starved, for example, for 1-4 days before harvesting or for 1-2 days (24-48 hours) after washing.

An embodiment of the method produces larvae with 50% protein content and 25% fat content. BSF larvae are incubated in a low moisture substrate (e.g., a substrate with a moisture content in the range of 0.3-0.45 g/ml) at a density of larvae of 45,000-133,000 larvae per square meter. The larvae are fed a larval feed having low fat content (5%-15%), such as beer mash, which has a fat content of about 15%. Larvae are starved for 3-4 days prior to harvesting and are starved for another 1-2 days (24-48 hours) by placing them in water after harvesting.

Harvesting Mature BSFL

To isolate mature BSFL from the larval incubation container, the incubated BSFL with the incubation mixture (i.e., inert substrate material and feed) are exposed to an air flow with frequent mixing or addition of dry sieved inert substrate material. The mixture is mixed thoroughly to dislodge clumps. The ideal density for separation of the BSFL from the incubation mixture is 0.27-0.3 g/ml. Above 0.3 g/ml, the incubation material becomes too wet and clogs the pores or mesh of the separator, which can be a sieve, a tumbler or similar tool. In an embodiment, a shaker or rotary sieve used to separate earth worms in vermicomposting can be used.

The BSFL are separated from the incubation mixture using the separator, e.g., sieve. In one embodiment, the sieve has pores of about 3.2 mm×3.2 mm (⅛ inch×⅛ inch). In another embodiment, the sieve has pores of about 2 mm×2 mm.

The yield of the above-described method for mass-rearing BSFL is about 4.5-6 kg wet weight BSFL per m² incubator in 10-12 days.

The food conversion ratio (FCR) depends on the quality of the food. With beer mash used as the larval feed, FCR=0.98.

The water consumption is about 42.5 L per m² incubator in 10 days or about 9.4 L of water per 1 kg of wet weight BSFL biomass.

5.3. Uses for Mass-Reared Dipteran Larvae

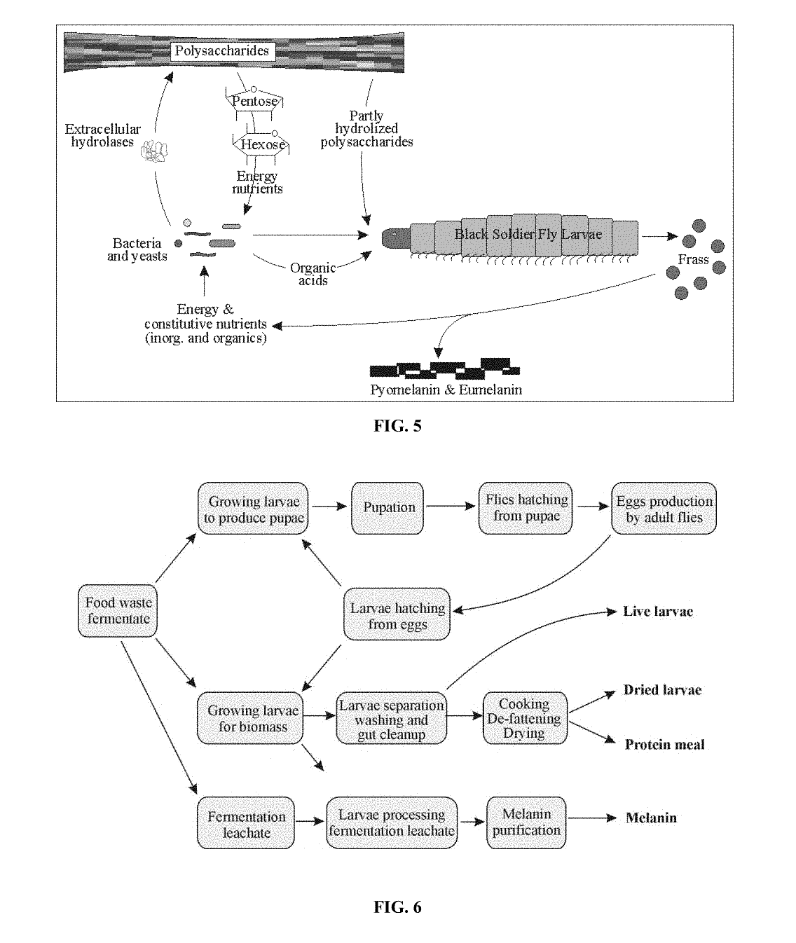

The methods and systems disclosed herein can be used to mass-rear large numbers of dipteran larvae that can be used to convert raw materials (e.g., food waste, residues of food industry, gardening and agriculture vegetal waste) into live larvae biomass, larvae feed, dried larvae, protein meal and melanin (FIG. 6).

Mass-reared dipteran larvae such as BSFL can be used for the processing and disposal of food waste products. BSFL are grown according to the mass-rearing methods disclosed herein. Food waste is the cheapest source of nutrients and fermented food waste is excellent for producing both leachate, which can be used produce melanin, melanin-associated proteins and/or fertilizer, and fermented solid residue, which can be used to grow the BSFL from the first instar hatchling larval stage to the last instar, largest size, white larval stage. The large white larvae are grown to a sufficient size to withstand, for at least two weeks (and up to 6 months), the harshness of conditions in secondary processing bioreactors. Using the methods disclosed in U.S. Pat. No. 8,815,539, approximately 300-400 kg dry weight food waste produces about 100-130 kg of last instar larvae (about a 3:1 ratio of food waste to larvae produced).

Food waste fermentate can be fed to growing dipteran larvae that have been produced using the present methods to produce pupae. Adult flies hatching from the pupae are used to produce eggs that can be used in to produce additional larvae for the methods disclosed herein (FIG. 6, top row).

As disclosed in U.S. Pat. No. 8,815,539 and WO 2014/197708 A1, BSFL are grown in large quantities and used to digest microbes (e.g., bacteria) present in the primary leachate and convert it to secondary leachate. Because of this fermentation process, the concentration of melanin goes from a very low percentage in each microbe or bacterium to a highly-concentrated product in the secondary leachate as the microbes are digested by the BSFL. This large-scale, bio-concentration method allows production of useful products including, but not limited to, melanin, melanin-associated proteins, fertilizers and products derived from insect bodies such as animal feed, protein, fats, biodiesel and chitin. For example, chitin can be extracted, using methods well known in the art, from both the pupal shells that are left behind after eclosion of BSF pupae into the adult fly stage, and the fly carcasses left behind after the adult flies mate and die.

According to the method disclosed herein, BSFL are grown to the large, late instar white larval stage because in the late instars, they are of sufficient size to withstand for at least two weeks (up to 6 months) the harshness of conditions in melanin producing incubators. It takes 1 kg of larvae approximately 2 weeks to convert 1 L of primary leachate and molasses into 1 L of secondary leachate containing 2.5-10 g melanin.

Mass-reared BSFL can be used in methods for producing melanin, melanin-associated protein and/or inorganic fertilizers from fermentation leachates or from low-cost nutrient-rich solutions (U.S. Pat. No. 8,815,539; WO 2014/197708 A1). According to these methods, polysaccharides are partly converted into natural melanins or inorganic fertilizer, which are difficult to biodegrade and hence accumulate in the bioreactors. These methods can employ, as a source of nutrients, leachates produced from food waste and sugars-rich liquid waste of the food industry. These leachates can be used raw or they can be augmented with low-cost sugar-rich solutions such as molasses, hydrolyzed cellulose or starch. The method is inexpensive and does not require the use of expensive chemically-defined culture media.

FIG. 5 shows an overview of the methods disclosed in U.S. Pat. No. 8,815,539 and WO 2014/197708 A1, which are used to convert media rich in sugars and polysaccharides (comprised in primary leachate) in solutions with high concentration of melanin, including but not limited to pyomelanin (comprised in secondary leachate).

The methods disclosed in U.S. Pat. No. 8,815,539 and WO 2014/197708 A1, can be used to convert raw materials (e.g., food waste, residues of food industry; gardening and agriculture vegetal waste) into larva biomass and melanin, including but not limited to pyomelanin. Bacteria from the mixed culture produce extracellular cellulases which hydrolyze cellulose and hemicellulose to sugars (hexoses and pentoses). Along with other sugars present in the primary leachate, these added sugars are used by microorganisms (e.g., bacteria and fungi) as a source of energy and carbon. During growth, microorganisms take up nutrients and produce biomass and secondary metabolites (mostly organic acids, alcohols and amines). These microorganisms also produce melanin. BSFL grind partly digested polysaccharides to take up digestible organics and microbial biomass. They also consume microbial biomass and metabolites directly from liquid. BSFL do not digest cellulosic polysaccharides, but they grind larger particles into finer particles making them more prone to enzymatic hydrolysis. BSFL eliminate unused food in the form of a finely ground suspensions called frass (i.e., 20-200 μm diameter colloids), thus returning nutrients (C, N, P, S, etc.) and bioavailable energy to the medium. This recycling of nutrients and the removal of toxic metabolites make microorganisms divide indefinitely as long as the primary carbon and energy sources are available. As they grow, microorganisms continuously produce polyphenols (such as melanin), but neither BSFL nor microorganisms digest polyphenols efficiently. In this system melanins are not recycled, but they accumulate.

### 6. EXAMPLES

6.1. Example 1: Mass-Rearing of BSFL in a Nursery

BSFL eggs of are produced in a nursery, such as the embodiment shown in FIG. 3. Nurseries can be scaled up or down using methods known in the art to suit a wide range of production schemes and can have controlled environmental conditions. Air is continuously provided, e.g., with a fan or pump to provide O₂ to the larvae, to remove toxic volatile chemicals (such as ammonia, organic amines and sulfide) and to help microbial respiration. BSFL derived from BSF adults are propagated in the nursery (FIG. 3) under controlled conditions of temperature, illumination, humidity and food availability. Eggs laid by BSF adults are hatched in a 30° C. incubator or nursery and then transferred to the mass-rearing container.

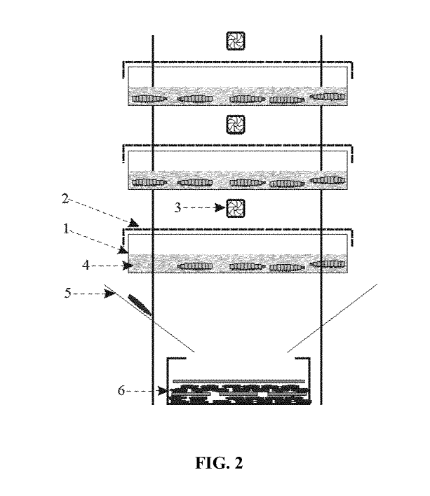

Pupae were produced using an embodiment of the system for propagating dipteran larvae shown in FIG. 2. FIG. 2 shows an embodiment of a system for propagating dipeteran larvae, e.g., black soldier fly larvae (BSFL). This embodiment of the system was directed to harvesting pupae and includes a pupae harvesting collection box for harvesting pupae (6). Systems for propagating can be scaled up or down to suit a wide range of the larval incubation container dimensions and production schemes. Larval incubation container (1). Mosquito net (2) to limit access of nuisance arthropods (e.g., insects) such as gnats and houseflies to the container. Aeration fan (3). Feed mixture (4). Larvae harvesting funnel (5). Pupae harvesting collection box (6).

The system comprised three larval incubation containers, 0.5 m² each, at 20° C., fed with 1.5 kg DW (dry weight) of beer mash. The system produced 0.75 kg of pre-pupae in one week. The larvae were allowed to pupate in a dry place, between wood boards, and kept in a dark pupae harvesting collection box (dark storage bin) at 20° C. for two weeks until flies started to hatch from pupae.

The pupae harvesting collection box was transferred in a fly tent (60 cm wide, 60 cm deep and 1.2 m high) with one water source and maintained in a room with natural light illumination plus one light bulb to extend the duration of light exposure to 12 hours per day. The moisture level in the room was kept at 60-80% relative humidity using a fogger mister. The temperature was 32+/−2° C. during the day and 25+/−5° C. during the night.

Flies hatched from pupae for approximately three weeks and the hatching success was approximately 35% in the first three weeks.

Flies mated, and the females laid eggs in the egg laying substrates (FIG. 3) provided. Egg clutches were harvested twice a week for 4 weeks. The efficiency of producing eggs was approximately 25% (egg clutches relative to the number of adult flies), with approximately 400-500 eggs per clutch.

The egg laying substrates containing the egg clutches were collected and transferred in hatching boxes with sealed lids (0.25 m² surface area and 15 cm high). Each box's interior and the egg laying substrates were sprayed with a total amount of approximately 10 ml of a saturated solution of methyl paraben. Each hatching box also contained a total amount of approximately 10 g of fine dust of wheat bran as initial newborn feed. The egg boxes were incubated in a room at 30+/−2° ° C. for one week.

After one week, the box was opened and the box, the lid and the egg laying substrates were washed with a mister into a water tank containing with warm water at a temperature of approximately 25-30° C.

The mixture of larvae and wheat bran feed was harvested on a filter of fine nylon fabric with 0.5-0.1 mm pores, producing a wet cake consisting of larvae, wheat bran and water. For each egg hatching box, the yield of wet cake was approximately 100 g per week. The wet cake was suspended in 6 L of warm water at approximately 25° C.

Larval incubation containers were prepared. Each larval incubation container had 0.5 m² surface area and was 20 cm high. The initial growth mixture consisted of the following: 3 kg of saw dust sieved twice to less than 3.2 mm particle size, 50 g of wheat bran sieved once to <3.2 mm particle size and 6 L of water/larvae suspension containing 100 g of larvae cake

The mixture was mixed thoroughly and placed in an incubator at 30° C. under an aeration fan.

On day 2, approximately 250 ml of water was added to compensate for water loss by evaporation.

On day 4, approximately 250 g of beer mash powder (dried, ground and sieved to <3.2 mm particle size) was added, plus 250 ml of water and 250 ml of a leachate (also referred to herein as “primary leachate”) produced by food waste fermentation. Methods of producing primary leachate are known in the art (see, e.g., U.S. Pat. No. 8,815,539 and WO 2014/197708 A1). The bedding material was thoroughly mixed.

On day 5, more water was added to compensate for evaporation and the bedding material was mixed thoroughly.

On day 6, 500 g of beer mash powder was added, 250 g of water and 250 ml of primary leachate. Sufficient water was then added to produce a mixture with approximately 0.45-0.5 g/ml density.

The procedure was repeated on days 7 through 10, by continually increasing the amount of feed (beer mash powder and primary leachate). Each 0.2 m² larval incubation container (growth box) was given approximately 3 kg of dry beer mash and 3 L of primary leachate. Each day the mixture was mixed twice thoroughly.

On day 10, the feeding was stopped. The mixture was thoroughly mixed at least three times a day and allowed to dry for two days to reach a density of approximately 0.3 g/ml.

On day 12, the mixture was processed in a rotary drum with ⅛-inch (˜3.2 mm) pore size to separate the larvae from the bedding material.

The separated larvae were still associated with some of the bedding material. The amount of bedding material remaining was approximately 3.5 kg per 0.5 m² larval incubation container. The larvae were mixed thoroughly with 3.5 kg of dry wood flour (sieved twice with a 3.2 mm particle size sieve) and separated again with the rotary drum separator to remove the remaining bedding material. This yield approximately 3 kg of live larvae.

The live larvae (3 kg) were transferred to a container with 10 cm deep water. The density of the live larvae was approximately 3 kg of larvae per 0.25 m². The live larvae in the container were maintained at room temperature for one day (day 13).

On day 14, the larvae were washed, drained and then frozen.

Frozen larvae can be thawed whenever they are needed. The frozen larvae were thawed, cooked for 10 min in boiling water with 0.1% ascorbic acid and then dried in an airflow at 65° C. to constant weight for 1.5-3 hours.

The efficiency of producing live larvae was approximately 6 kg of larvae per m² of larval incubation container incubated for 12 days. Approximately 9 kg of live larvae were produced per 1 kg of pupae used in the method of mass-rearing disclosed herein. The food conversion ratio was approximately 1 (i.e., 1 kg of dry beer mash per 1 kg of live larvae biomass)

The yield was approximately 16-20% of dry larvae biomass relative to the wet larvae biomass. Chemical analysis of the dry larvae contained 3.76% moisture, 49-55% crude protein, 12.48% carbohydrates, 18.5-25.8% fat, 480 calories per 100 g, 8.8% ash and 8% crude fiber.

The BSFL biomass that was de-fattened and turned into protein meal contained 3.8% moisture, 55.3% protein, 9.7% fat, 19.8% carbohydrates, 11.3% ash, 388 calories per 100 g and 10.4% crude fiber. The efficiency of turning live larvae into protein meal was approximately 15-16%.

A sample of methods that are described herein are set forth in the following numbered paragraphs:

1. A method for mass-rearing dipteran flies comprising:

providing:
a larval incubation container,
inert substrate material,
a larval feed,
dipteran fly larvae, and
water;
grinding and/or sieving the inert substrate material to obtain substrate material particles having largest diameters of ≤3.2 mm;
grinding and/or sieving the larval feed to obtain larval feed particles having largest diameters of ≤3.2 mm;
drying the inert substrate material;
adding dry inert substrate material to the larval incubation container;
adding the larval feed to the larval incubation container;
mixing the larval feed and the dry inert substrate material in the larval incubation container; adding the dipteran fly larvae to the larval incubation container;
adding water to the dipteran fly larvae, the larval feed and dry inert substrate material in the larval incubation container to obtain a ratio, wherein the ratio ranges by weight from 1.5 parts water:1 part dry inert substrate material plus the larval feed to 2.5 parts water:1 part dry inert substrate material plus the larval feed;
incubating the dipteran larvae in the larval incubation container; and
harvesting the dipteran flies produced in the larval incubation container.

2. The method of paragraph 1, wherein the largest diameters of the substrate material particles and/or the larval food particles are between 2 mm and 3.2 mm

3. The method of paragraph 1, wherein the dipteran flies are black soldier flies (BSF).

4. The method of paragraph 1, wherein the harvested dipteran flies are larvae.

5. The method of paragraph 1, wherein the harvested dipteran flies are pre-pupae.

6. The method of paragraph 1, wherein the harvested dipteran flies are pupae.

7. The method of paragraph 1, wherein the harvested dipteran flies are adults.

8. The method of paragraph 1, wherein the inert substrate material is a ligno-cellulosic material.

9. The method of paragraph 1, wherein the dipteran larvae added to the larval incubation container are black soldier fly larvae (BSFL) that are 2-4 days old and weigh about 5-30 mg wet weight per larva.

10. The method of paragraph 4, further comprising, before the harvesting, starving the dipteran fly larvae for 2-4 days prior to the harvesting.

11. The method of paragraph 4, further comprising, after the harvesting, placing the harvested larvae in water and starving the harvested larvae for approximately 24-48 hours.

12. The method of paragraph 1, wherein the larval feed is a low-fat larval feed having a fat content of 5-15%.

13. A system for mass-rearing dipteran flies comprising:

a larval incubation container,
dry inert substrate material, wherein the dry inert substrate material comprises substrate material particles, wherein the largest diameters of the particles are ≤3.2 mm.
a larval feed,
dipteran fly larvae, and
means for separating the dipteran fly larvae from the substrate material.

14. The system of paragraph 13, wherein the largest diameters of the substrate material particles and/or the larval feed particles are between 2 mm and 3.2 mm

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

While embodiments of the present disclosure have been particularly shown and described with reference to certain examples and features, it will be understood by one skilled in the art that various changes in detail may be effected therein without departing from the spirit and scope of the present disclosure as defined by claims that can be supported by the written description and drawings. Further, where exemplary embodiments are described with reference to a certain number of elements it will be understood that the exemplary embodiments can be practiced utilizing either less than or more than the certain number of elements.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

What is claimed is:

1. A method for mass-rearing dipteran flies comprising:
providing:
a larval incubation container,
inert substrate material,
a larval feed,
dipteran fly larvae, and
water;
grinding and/or sieving the inert substrate material to obtain substrate material particles having largest diameters of ≤3.2 mm;
grinding and/or sieving the larval feed to obtain larval feed particles having largest diameters of ≤3.2 mm;
drying the inert substrate material;
adding dry inert substrate material to the larval incubation container;
adding the larval feed to the larval incubation container;
mixing the larval feed and the dry inert substrate material in the larval incubation container;
adding the dipteran fly larvae to the larval incubation container;
adding water to the dipteran fly larvae, the larval feed and dry inert substrate material in the larval incubation container to obtain a ratio, wherein the ratio ranges by weight from 1.5 parts water:1 part dry inert substrate material plus the larval feed to 2.5 parts water:1 part dry inert substrate material plus the larval feed;
incubating the dipteran larvae in the larval incubation container; and
harvesting the dipteran flies produced in the larval incubation container.

2. The method of claim 1, wherein the largest diameters of the substrate material particles and/or the larval feed particles are between 2 mm and 3.2 mm.

3. The method of claim 1, wherein the dipteran flies are black soldier flies (BSF).

4. The method of claim 1, wherein the harvested dipteran flies are larvae.

5. The method of claim 1, wherein the harvested dipteran flies are pre-pupae.

6. The method of claim 1, wherein the harvested dipteran flies are pupae.

7. The method of claim 1, wherein the harvested dipteran flies are adults.

8. The method of claim 1, wherein the inert substrate material is a ligno-cellulosic material.

9. The method of claim 1, wherein the dipteran larvae added to the larval incubation container are black soldier fly larvae (BSFL) that are 2-4 days old and weigh about 5-30 mg wet weight per larva.

10. The method of claim 4, further comprising, before the harvesting, starving the dipteran fly larvae for 2-4 days prior to the harvesting.

11. The method of claim 4, further comprising, after the harvesting, placing the harvested larvae in water and starving the harvested larvae for approximately 24-48 hours.

12. The method of claim 1, wherein the larval feed is a low-fat larval feed having a fat content of 5-15%.

13. A system for mass-rearing dipteran flies comprising:
a larval incubation container,
dry inert substrate material, wherein the dry inert substrate material comprises particles, wherein the largest diameters of the substrate material particles are ≤3.2 mm,
a larval feed, wherein the largest diameters of the larval feed particles are ≤3.2 mm,
dipteran fly larvae, and
means for separating the dipteran fly larvae from the substrate material.

14. The system of claim 13, wherein the largest diameters of the substrate material particles and/or the larval feed particles are between 2 mm and 3.2 mm.
